(19) United States
(12) Patent Application Publication
(10) Pub. No.: US 2003/0139368 A1
(43) Pub. Date: Jul. 24, 2003

(54) Antithrombotic and/or anti-inflammatory composition combining purine activity and NSAID activity

(76) Inventors: Philippe Gorny, Paris (FR); Bertrand Mailliard, Paris (FR)

Correspondence Address: IP DEPARTMENT OF PIPER RUDNICK LLP, 3400 TWO LOGAN SQUARE, 18TH AND ARCH STREETS, PHILADELPHIA, PA, 19103, US

(21) Appl. No.: 10/360,280

(22) Filed: Feb. 7, 2003

Related U.S. Application Data
(63) Continuation of application No. PCT/FR01/02580, filed on Aug. 8, 2001.

(30) Foreign Application Priority Data
Aug. 8, 2000 (FR) 00/10435

(51) Int. Cl. A61K 31/7076; A61K 31/54; A61K 31/522; A61K 31/415; A61K 31/21; A61K 31/192

(52) U.S. Cl. 514/45; 514/46; 514/406; 514/570; 514/161; 514/509; 514/227.5; 514/263.31

(57) ABSTRACT

A method which treats inflammation or thrombosis and includes administering to a mammal a therapeutically effective amount of a composition including a purine and an NSAID as active ingredients. A method which prevents inflammation or thrombosis and includes administering to a mammal a therapeutically effective amount of a composition including a purine and an NSAID as active ingredients. An anti-inflammatory or anti-thrombotic composition which includes one or more molecules of purine covalentlylinked to one or more molecules of NSAID, or optionally, by at least one spacer arm intermediary.

### RELATED APPLICATION

This is a continuation of International Patent Application PCT/FR01/02580 filed Aug. 8, 2001, which claims priority of French Application No. 00/10435 filed Aug. 8, 2000.

### FIELD OF THE INVENTION

This invention combines purine activity and nonsteroidal anti-inflammatory drug (NSAID) activity in a composition intended to be used as an antithrombotic agent and/or anti-inflammatory agent.

### BACKGROUND

It is known that the lesion of a blood vessel leads to activation of the coagulation system and aggregation of blood platelets (or thrombocytes). The result is formation of a blood clot constituted by fibrin filaments in which platelets are confined. This clot blocks the wound which stops the bleeding and eventually allows return to normal blood circulation.

It can also occur that a clot (or thrombus) forms for no purpose in a blood vessel. Such a clot, constituted by platelet aggregates which are often deposited at the level of arterial atherosclerosis plaque can then cause a cardiac, cerebral or other infarction, or acute ischemia of an artery of the arm or leg. A weak blood flow rate increases the risk of appearance of a thrombus, especially in the venous network of the legs, and a blood clot detached from this thrombus can be carried to a pulmonary artery which it obstructs, thereby causing a pulmonary embolism.

Anticoagulants as well as inhibitors of platelet aggregation can be used in the prophylaxis of thromboses. The inhibitors of cyclooxygenase can inhibit notably the synthesis of thromboxane A₂ which possesses both aggregating and vasoconstrictive properties. The cyclooxygenase inhibitors are thus capable of constituting antithrombotic drugs of interest. Low-dose aspirin is presently used in this indication.

It is also known that nucleotides are involved in numerous physiological processes including vascular tonus, cardiac contractions and platelet aggregation.

In particular it is known that ADP plays a key role in the induction of platelet aggregation via the intermediary of certain P2 platelet receptors, in particular the receptors P2X1, P2Y1 and P2T. AMP acts notably via the intermediary of P1 receptors (in particular, the P1A2a receptors).

### SUMMARY OF THE INVENTION

This invention relates to a method of treating inflammation or thrombosis including administering to a mammal a therapeutically effective amount of a composition including a purine and an NSAID as active ingredients.

This invention also relates to a method of preventing inflammation or thrombosis including administering to a mammal a therapeutically effective amount of a composition including a purine and an NSAID as active ingredients.

This invention further relates to an anti-inflammatory or anti-thrombotic composition including one or more molecules of purine covalently linked to one or more molecules of NSAID, or optionally, by at least one intermediary spacer arm.

### DETAILED DESCRIPTION

We have now discovered that the combination of purine activity and nonsteroidal anti-inflammatory (NSAID) activity produces a potentiating synergic effect in the inhibition of platelet aggregation. This combination can be attained either by administration of a product acting on the purinergic receptors and an NSAID, or by administration of a product in which one or more purine molecules is bound by covalence to one or more NSAID molecules, possibly by the intermediary of at least one spacer arm. Such products are notably the products of formula I to be described below.

We can mention here as an example of purine activity either an agonist activity on the receptors involved in the inhibition of platelet aggregation and having natural AMP and/or adenosine ligands (for example, P1 type receptors) or an antagonist activity on the receptors involved in platelet aggregation (proaggregating receptors) and having ADP for natural ligand (for example, P2 type receptors).

We have also discovered that AMP used by itself possesses an antiaggregating activity, and AMP can also be used as active ingredient in the preparation of an antiaggregating drug.

It is also known that inflammation is the response of a vascularized living tissue to a local lesion (trauma, infection, irritation by chemical products, etc.). The inflammation of tissues is manifested notably by symptoms such as redness, swelling and pain. The lesion initially causes vasodilation accompanied by an augmentation of the vascular permeability, promoting the migration of leukocytes to the injured site. Various chemical mediators participate in the inflammatory reaction, among which we can cite in particular the metabolites of arachidonic acid (AA). These metabolites act locally on the blood vessels and on the injured site but they are rapidly destroyed. Arachidonic acid is a constituent of the phospholipids of the cell membrane. Under the action of various stimuli, the membrane phospholipases release AA which is then the object of metabolic transformations of which the two most important are linked to its use as substrate for cyclooxygenases (or Cox) and lipoxygenases (or Lipox). The cells producing AA during the inflammatory response are principally the leukocytes and platelets.

The cyclooxygenase pathway leads to the production of prostaglandins and thromboxanes, which have numerous biological activities which can depend on the cells producing them. Vasodilation, augmentation of vascular permeability, augmentation of painful sensations, induction of fever, etc. are among these biological activities.

The lipoxygenase pathway leads to leukotrienes which contribute to inflammation in various pathologies such as rheumatoid arthritis, asthma, psoriasis, gout, etc.

The most effective anti-inflammatory substances known at present act on the metabolism of arachidonic acid: the steroidal anti-inflammatory agents inhibit phospholipase while the nonsteroidal anti-inflammatory agents (NSAIDs) inhibit cyclooxygenase and thus inhibit the formation of prostaglandins and thromboxanes.

Two isoenzymes of cyclooxygenase are known to exist, i.e., Cox-1, which is expressed in a permanent manner, notably in the stomach, and Cox-2, an inducible form which is only expressed during inflammatory reactions. Cox-1 produces in the stomach prostaglandins which have the function of protecting the gastric mucosa against the ambient acidity. Most of the NSAIDs inhibit both Cox-1 and Cox-2, and, therefore, have unpleasant side effects such as deterioration of the gastric mucosa with risks of bleeding or formation of an ulcer. NSAIDs are now known which do not exhibit this drawback (for example, rofecoxib and celecoxid) because they are specific inhibitors of Cox-2.

We also know that certain purines, e.g., adenosine and AMP, are anti-inflammatory agents. Adenosine (endogenous or exogenous), for example, protects cells against certain oxidizing free radicals and inhibits neutrophilic leukocytes. AMP inhibits the migration of leukocytes during inflammation. Moreover, adenosine intervenes in the mechanism of action of methotrexate and sulfasalazine which are used in the treatment of rheumatoid arthritis. Adenosine also inhibits in humans the liberation of various proinflammatory cytokines such as IL-6, IL-8, IL-12 and TNF.

We have further discovered that the combination of purine activity and NSAID activity enables a potentiating synergic effect in the inhibition of the inflammatory reaction. This combination can be attained either by administration of a purine and an NSAID, or by administration of a product in which one or more purine molecules is bound by covalence to one or more NSAID molecules, optionally by the intermediary of at least one spacer arm. Such products are notably the products of formula I described below.

The invention, thus, utilizes a combination of a purine with an NSAID as active ingredients in a composition intended to combat thromboses and/or inflammation.

“Purine” is understood to mean especially purine-based nucleosides and nucleotides and, in particular, adenosine as well as the corresponding phosphates, notably AMP, ADP and ATP, guanosine, GMP, GDP, GTP, inosine as well as their mono-, di- and triphosphates, and their derivatives or analogues, notably their pharmaceutically acceptable salts (for example, hydrochlorides of nucleosides or nucleotides with an amine functional group, or alkaline salts of the nucleotides). “Purine” is more generally also understood to mean any substance capable of acting on the purine receptors, which are also referred to as purinergic receptors (notably P1 receptors sensitive to AMP and adenosine, and P2 receptors sensitive to ADP and ATP). Such substances are known or can be found by known methods. “Purine activity” is an activity obtained by the presence of a purine such as defined above. We can cite in particular among the purine analogues notably in the case of the preparation of an antiaggregating drug the agonists of the type P1 receptors and the antagonists of the type P2 receptors. Such agonist or antagonist products are known: see especially the site www.sigma-aldrich.com, heading RBI. Moreover, the search for such agonists or antagonists can be performed according to known methods by simple routine experiments. It is obviously understood that neither ADP nor its agonists are used as active ingredients in the preparation of an antiaggregating drug by combination of a purine and an NSAID.

Generally speaking in this application “derivatives” refer to products obtained by modifying a chemical functional group or an atom or a group of atoms of an active product, and which have a physiological activity of the same type as the active product. As examples, the derivatives of active products having acid functional groups can be notably the salts (for example, sodium salts or salts of other alkaline metals, or salts formed with amines, e.g., piperazine salts or lysine salts), or the esters formed by the acids with alcohols, or the amides formed by these acids with amines; the derivatives of active products having amine functional groups are notably the amides and the addition salts formed by these amines with the acids; the derivatives of active products having alcohol functional groups are notably the esters formed by the alcohols with the acids.

The nonsteroidal anti-inflammatory drugs or NSAIDs constitute a known class of anti-inflammatory agents which have many properties in common: a cyclooxygenase inhibition activity which gives them the capacity to inhibit the synthesis of prostaglandins. The NSAIDs have other properties in common: decoupling of oxidative phosphorylation, modifications of the intracellular movements of calcium ions, activation of the synthesis of inducible NO synthase, action on the kappa nuclear factors, and the like. It is possible that one or more of these properties are responsible for the potentiating effect of the NSAIDs on purines, but it is also possible than other known or unknown properties are involved.

Among the nonsteroidal anti-inflammatory drugs we can cite, e.g. (see especially THE MERCK INDEX, 12^{th} edition, Therapeutic Category and Biological Activity Index):

aminoarylcarboxylic acid derivatives such as: enfenamic acid, etofenamic acid, flufenamic acid, isonixin, meclofenamic acid, mefenamic acid, niflumic acid, talniflumate, terofenamate, tolfenamic acid;

arylacetic acid derivatives such as: aceclofenac, acematacin, alclofenac, amfenac, amtolmetin guacile, bromfenac, bufexamac, cinmetacin, clopirac, diclofenac, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesine, zomepirac;

arylbutryic acid derivatives such as: bumadizon, butibufen, fenbufen, xenbucin;

arylcarboxylic acid derivatives such as: clidanac, ketorolac, tinoridine;

arylpropionic acid derivatives such as: alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprofen, pirprofen, pranoprofen, protizinic acid, methiazinic acid, suprofen, tiaprofenic acid, ximoprofen, zaltoprofen, maproxen;

salicylic acid derivatives such as: acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, methyl salicylate, phenyl salicylate, salacetamide, acetic [2-(aminocarbonyl) phenoxy] acid, salicylsulfuric acid, salsalate, sulfasalazine, aspalatone; as well as the nitric esters of the salicylates or aspirin such as 2-(2-nitroxy)-butyl 2-acetoxybenzoate and 2(2-nitroxymethyl) phenyl 2-acetoxybenzoate;

other carboxylic acid derivatives such as: ε-acetamidocaproic acid, 3-amino-4-hydroxybutyric acid;

pyrazole or pyrazolone derivatives such as: defenamizole, epirazole, apazone, benzpiperylon, feprazone, suxibuzone, bumadizone, clofezone, kebuzone, mofebutazone, proxifezone, morazone, oxyphenbutazone, phenylbutazone, pipebuzone, propyphenazone, pyrazinophenazone, ramifenazone, thiazolinobutazone, tolmetin, antipyrine, noramidopyrine, dipyrone, azapropazone, celecoxib;

thiazinecarboxamide derivatives such as: ampiroxicam, droxicam, isoxicam, lomoxicam, piroxicam, tenoxicam;

other anti-inflammatory drugs such as: S-adenosylmethionine, amixetrine, bendazac, benzydamine, α-bisabolol, buculome, difenpiramide, ditazol, emorfazone, fepradinol, guaiazulene, nabumetone, nimesulide, oxaceprol, paranyline, perisoxal, proquazone, tenidap, zileuton, rofecoxib;

as well as NSAID nitrogen monoxide donor derivatives such as the nitric esters and the nitro or nitroso derivates described in the patents and patent applications EP 0 670 825, U.S. Pat. No. 5,700,947, WO 95/30641, U.S. Pat. No. 5,703,073, U.S. Pat. Nos. 6,043,232 and 6,043,22, the contents of which are incorporated herein by reference.

In the above list of NSAIDs, the common international denomination indicates the active base ingredients as well as their immediate derivatives that can be used pharmaceutically (e.g., the acids as well as their salts).

The NSAIDs, including the carboxylic group NSAIDs, are known products described notably in THE MERCK INDEX, 12^{th} edition, the content of which (including the data and references pertaining to NSAIDs) is incorporated herein by reference.

Among the anti-inflammatory drugs that can be used, it can be of value to select a product that selectively or preferentially inhibits Cox-2 (e.g., rofecoxib, celecoxib, nabumetone).

The active ingredients of a composition obtained in accordance with the invention can be presented separately, each ingredient being in a suitable pharmaceutical form, and brought together in a single package. However, in order to facilitate simultaneous administration of the active ingredients, it is generally preferred to prepare the composition as a single pharmaceutical form containing both active ingredients, possibly as well as a pharmaceutically suitable excipient.

It is understood that a product that has both purine activity and NSAID activity should be considered to itself be a combination having the two types of activity, and as such can be used in accordance with the invention as a single active ingredient. For example, a purine and an NSAID can be combined by establishing a chemical bond between the two molecules. It is possible notably to amidify an amine function of the purine, or esterify one or more alcohol functional groups of the product with purine activity with an acid group present in an NSAID with a carboxylic functional group such as, e.g., acetylsalicylic acid, mefenamic acid, diclofenac, naproxen, ibuprofen, sulindac, etc. It is possible notably to amidify an amine function of the puric base of the purine, or esterify one or more alcohol functional groups of the purine ose (nucleoside or nucleotide). One thereby obtains an amidification product that possesses both a purine activity and an NSAID activity. Examples of such products are the products of formula I described below.

The composition obtained in accordance with the invention can be administered via the oral, sublingual, nasal, pulmonary, rectal or parenteral (e.g., intravascular, intramuscular, transcutaneous, intra-articular) route.

For this purpose, the composition can be in any form enabling administration via the oral route (in particular in the form of capsules, solutions or emulsions for oral administration, powders, gels, granules, tablets or compressed tablets), via the nasal route (e.g., solutions to be administered in the form of drops or sprays), via the pulmonary route (solutions in pressurized aerosol containers), via the rectal route (suppositories), via the cutaneous route (e.g., creams, unguents or transdermal devices, referred to as patches), by injection (injectable solutions, lyophilized powders to be reconstituted as injectable solutions) or via the transmucosal route such as, e.g., via the sublingual route (solutions in pressurized containers or tablets for buccal dissolution).

These pharmaceutical forms are prepared in a conventional manner and can contain appropriate conventional excipients and vehicles.

The composition of the invention can be prepared, e.g., in a pharmaceutical form enabling administration to a subject requiring such a drug, e.g., a human subject, of about 10 to about 1000 mg of purine per day and also enabling administration of an adequate dose of NSAID, e.g., a dose of about 10 to about 3000 mg of NSAID per day.

As an example, a dose of abut 50 to about 500 mg of AMP and about 10 to about 1000 mg per day can be administered to a human adult. The AMP can be replaced, notably, by equivalent quantities of adenosine. If it is desired to replace AMP with another purine and/or aspirin with another NSAID, the dose ranges stated above can easily be adjusted by replacing a given dose of AMP with an equivalent dose of another purine and/or replacing a given dose of aspirin with an equivalent dose of another NSAID. Such an equivalent dose can be determined, e.g., using any conventional anti-inflammatory test. A purine dose equivalent to a given dose of AMP is, e.g., a dose capable of having a comparable antiaggregating effect in the tests described below in the experimental part.

It is, of course, understood that the dose can be adjusted especially in relation to the treated subject's body weight.

The composition obtained according to the invention can be administered as an antithrombotic agent and antiaggregating agent notably in the treatment of angina pectoris, circulatory insufficiencies of the legs, for the prevention of infarctions in atherosclerotic patients and also for the purpose of preventing the recurrence of infarctions, especially cardiac and cerebral infarctions.

The composition obtained according to the invention can also be administered as an anti-inflammatory agent in all of the pathologies in which it is desired to inhibit or limit the inflammatory reaction, notably in the treatment of arthroses, rheumatoid arthritis, tendonitis, attacks of gout, inflammatory diseases of the intestine, dysmenorrhea, posttraumatic edema, ankylosing spondylarthritis and in cases in which it is desired to combat pain and fever.

It is also known that after an angioplasty, the forced dilation of an artery by a balloon is the equivalent of a parietal and endothelial trauma (the endothelium being the internal coating of the artery), which triggers an automatic cell repair mechanism. The smooth muscle cells, subjacent to the endothelium, secrete various growth factors which have an essential promoter role. The danger comes from an excess of parietal reaction due to an inflammatory type reaction, in relation to a zone which is itself abnormal, with thickening of the vascular wall, inducing the reconstitution of a new stenosis (or restenosis) due to an excess of cicatrization. The result is that about 30% of angioplasties are restenosed after 6 months. It is then necessary to redilate or have recourse to surgery. Various methods appear to be able to reduce the incidence of restenoses: the use of metal stents (springs that keep the vessel open which are put in place after dilation), in situ radiotherapy, the release or provision on the site of anti-inflammatory substances that inhibit restenosis. This last method is justified by the fact that the triggering of the parietal reaction is a phenomenon that immediately follows the trauma of the dilation.

The combination of a purine and an NSAID (e.g., the combination of aspirin with adenosine or sulindac with adenosine) administered immediately after an angioplasty can inhibit or reduce restenosis.

As stated above, it is possible to replace the combination of a purine and an NSAID by a single product in which a purine or purine analogue is bound by covalence to an NSAID, e.g., a product of formula I as described below.

The invention also includes new products comprising an NSAID and a purine bound by covalence to the NSAID, optionally by the intermediary of at least one spacer arm.

These products are notably those that respond to formula I

(A-)ₘ(X)ₚ(—B)ₙ (I)

wherein A is the residue of an NSAID molecule, B is the residue of a purine and X represents either a covalent bond between A and B, or a spacer arm linking at least one A residue with at least one B residue, m is a whole number ranging from 1 to 3, n is a whole number ranging from 1 to 3, and p represents zero or a whole number equal at most to the larger of the numbers m and n. It is possible, depending on the case, to either graft one or more A and/or B residues on a single spacer arm, or graft one or more A-X— groups on a B residue (and then m=p and n=1), or graft one or more —X—B groups on an A residue (and then n=p and m=1). When p=zero, either one or more A residues are linked to a B residue (and n=1), or one or more B residues are linked to an A residue (and m=1).

The products of formula I can be used in the form of salts, particularly in the form of alkaline metal salts such as sodium or potassium salts. These salts are, e.g., those of the phosphate groups if they are present, the phenolic groups (in the case of salicylic acid) and the like. It is also possible to use the products of formula I, where appropriate, in the form of addition salts (e.g., in hydrochloride form) when these products contain an amine group.

The bonds between the spacer arm and the A and B residues are covalent bonds. The chemical groups creating the link between A and B (when p=zero), or between A and X or between X and B (when p is other than zero), are, e.g., carboxylic ester, carboxylic amide, thiocarboxylic ester or thiocarboxylic amide groups.

In formula I, A can represent, but is not limited to, the acyl residue of an NSAID possessing a carboxylic group (the NSAID would, thus, have the formula A-OH) and B can represent the residue of a purine base nucleoside or nucleotide bound to X, or bound to A (in the case of absence of spacer arm), by the intermediary of the nitrogen of a primary amine of the purine base and/or by the intermediary of the oxygen of a hydroxyl group of said purine base nucleoside or nucleotide. For example, one or more A or A-X— groups can be linked to B by the intermediary of the oxygen of the primary alcohol of said nucleoside and/or by the intermediary of the oxygen of at least one secondary alcohol of said nucleotide. In these cases, the purine from which B is derived has as its formula BH.

In formula I, the nucleoside or nucleotide is notably a ribonucleoside or ribonucleotide. The purine can be selected from among adenosine, guanosine and inosine, as well as the corresponding 5′-monophosphates, -diphosphates and -triphosphates.

The spacer arms can be notably bivalent residues of bifunctional aliphatic compounds (i.e., compounds having at each of their ends reactive functional groups enabling formation of covalent bonds with A and with B). These compounds can be, e.g., compounds that possess both an amino group and a carboxylic (or thiocarboxylic) group, or rather compounds that possess both an amino group and a hydroxyl group.

In formula I, the group X (leaving aside these end functional groups) can represent a divalent aliphatic group possibly interrupted by one or more —O— or —S— heteroatoms or by one or more NH— or —CO—NH— heteroatomic groups.

The spacer agents, i.e., the compounds capable of yielding, after reaction with the purine and NSAID, products of formula I in which A and B are linked by spacer arms, are, e.g., alpha-, beta- or gamma-amino alkanecarboxylic acids, in particular the natural alpha-amino acids such as glycine, alanine, valine or leucine, or peptides, notably dipeptides or tripeptides.

The spacer agents can also be hydroxycarboxylic acids such as lactic or glycolic acids, the aldonic acids (gluconic, mannonic, galactonic, ribonic, arabinonic, xylonic and erythronic acid) and the corresponding lactones or dilactones (e.g., lactide, glycolide, delta-glucolonactone, delta-valeronactone), or the aldaric acids.

The functional groups possibly present on the spacer arm and not involved in the bond with an A or B element can be used for grafting other A and/or B residues to obtain compounds of formula I for which m and/or n is greater than 1. This is the case, for example, with the hydroxyl groups of the hydroxy acids, the second carboxylic group of the amino diacid carboxylic acids, the second amino group of the diaminated amino acids, the hydroxyl group of the hydroxylated amino acids.

The classic methods of organic synthesis are used to prepare the compounds of formula I. For example, to prepare amides or esters, one can react a carboxylic compound (NSAID or spacer agent) in the form of a carboxylic (or thiocarboxylic) acid halide or in the form of a mixed anhydride or in the form of an activated ester, e.g., an ester of p-nitrophenyl. The acid can also be activated by means of a coupling agent such as dicyclohexylcarbodiimide.

Since the compounds of formula I comprise residues of nucleosides or nucleotides, they can be prepared using in particular the methods known in nucleic acid chemistry, described for example in the publication by Kochetkoc and Budovskii, Organic Chemistry of Nucleic Acids, Plenum Press, 1971 (2 volumes), the contents of which are incorporated herein by reference.

When the compounds from which derive A, B or X of formula I comprise multiple functional groups capable of reacting that it is appropriate to operate either using the reagents in stoichiometric proportions (according to the number of precursor products of A and/or B that it is desired to react), or by temporarily protecting the reactive functional groups that one does not want to react. For this, use is made of temporary protection methods for the reactive functional groups. These temporary protection methods are well known, notably those that were developed in research focused on peptide synthesis. For example, —NH₂ groups can be protected by carbobenzoxy, phthaloyl, t-butoxycarbonyl, trifluoroacetyl or toluenesulfonyl groups; carboxylic groups can be protected in the form of benzyl esters, tetrahydropyranyl esters or t-butyl esters; alcohols can be protected in the form of esters (e.g., acetates), in the forms of tetrahydropyranyl ethers, benzyl ethers or trityl ethers, or in the form of acetals (including in the form of acetonides in the case of vicinal glycols). The reactions of protection and possible deprotection of various chemical groups are known and described, e.g., in the publication Advances in Organic Chemistry, Methods and Results, Vol. 3, Interscience Publishers (1963), pages 159 and following and pages 191 and following, as well as in the publication by T. W. Green, Protective Groups in Organic Synthesis, Wiley-Interscience Publication (1991). The contents of these publications are incorporated herein by reference.

The phosphatation or dephosphatation reactions of the primary alcohol of the nucleotides or nucleosides can be implemented using natural enzymes (e.g., phosphatases, phosphokinases).

Among the products of formula I can be cited in particular those responding to formula Ia:

A-B (Ia),

wherein A and B are defined as above. A preferably represents the acyl residue of an NSAID possessing a carboxylic group, the bond with B being made, e.g., by formation of an amide or an ester with an amine or alcohol functional group, respectively, of the purine of formula BH.

Among the products of formula I or Ia, we can cite notably the amides and esters formed with the acyl A residues of salicylic acid, acetylsalicylic acid, diclofenac, ibuprofen, naproxen or sulindac, and with the B residues derived from adenosine or AMP.

It is of particular value to select among the products of formula I those that have a potentiating synergic effect in relation to their purine and NSAID constituents. Such products can be selected by simple routine experiments.

It should also be noted that the products of formula I or Ia in general have improved gastric tolerance compared to the NSAIDs from which they are derived.

The products of formula I or Ia are administered in the same pharmaceutical forms as those described above, and at equivalent doses, taking into account the respective proportions of the molecules derived from purine and NSAID in the product of formula I or Ia under consideration. The doses to be administered can be determined by routine experiments using tests known from research on anti-inflammatory activity or antiaggregating activity.

The indications presented above for the products of formula I or Ia also apply, in a general manner, to all products comprising an NSAID and a purine, linked by covalence, optionally by the intermediary of at least one spacer arm.

The following examples illustrate selected aspects of the invention.

### EXAMPLE 1

### Preparation of the Product of Formula A-NH—Y, in which A is the Acyl Residue of Acetylsalicylic Acid and Y is the AMP Residue Amputated of its Primary Amine, the NH Group of the Formula Representing the Residue of Said Primary Amine of AMP

This is, thus, the product of amidification of AMP by acetylsalicylic acid.

The starting products were acetylsalicylic acid (source: SIGMA) and AMP (or (5′-O-phosphate)adenosine), sodium salt (source: SIGMA).

0.5 g of AMP was dissolved in 14 ml of water at room temperature. 0.17 g of potassium carbonate (K₂CO₃) was added, then 0.25 g of 2-acetyl benzoyl chloride was added, followed by 3 ml of dioxane to solubilize the latter ingredient. The mixture was then mixed for 12 hours at room temperature. The solvents were then evaporated.

The chlorides were eliminated by dialysis. The resultant product was then purified by silica gel chromatography, eluting with a 1:1:1 ethyl acetate/water/isopropanol mixture.

The phosphate group was salted by sodium and potassium ions.

The NMR spectra of the proton and phosphorus ³¹P were in agreement with the indicated structure.

Operating in a similar manner but replacing the potassium carbonate with sodium carbonate, we obtained the corresponding amide whose phosphate groups were salted by sodium ions.

### EXAMPLE 2

### Preparation of the Product of formula A-NH—Y, in which A is a Salicylyl Residue and Y is the AMP Residue (Amputated of its Primary Amine), the NH Group of the Formula is the Residue of Said Primary Amine of AMP

This is, thus, the product of amidification of AMP by salicylic acid.

The starting product was the product of Example 1, which was subjected to desacetylation in a basic medium. This product was dissolved in water and 3 equivalents of potassium carbonate K₂CO₃ were added at room temperature. At the end of a reaction time of 12 hours at room temperature, we obtained the product indicated in the title of this example.

The desacetylated product was purified by silica gel chromatography as in Example 1.

The phosphate and phenolate functions of this product were salted by sodium and potassium ions.

Replacing the potassium carbonate with sodium carbonate, we obtained the indicated product whose phosphate and phenolate functions were salted by sodium ions.

### EXAMPLE 3

### Preparation of a Product of formula A-NH—Y, in which A Represents a Salicylyl Residue, Y Represents an Adenosine Residue Amputated of its Primary Amine, and the NH Group of the Formula Represents the Residue of Said Primary Amine of Adenosine

This is, thus, the product of amidification of adenosine by salicylic acid.

a) adenosine 2′,3′,5′,O-triacetate:

5.34 g of adenosine was mixed with 11.4 ml of acetic anhydride dissolved in 25 ml of anhydrous pyridine. Agitation was performed for 12 hours at room temperature. The solvents were then evaporated. The residue was then taken up several times with ethanol. We obtained 6.97 g of white crystals.

b) adenosine N-(2-acetylsalicylyl) 2′,3′,5′,O-triacetate:

2.457 g of the product obtained in a) was dissolved in 26 ml of dichloromethane and 0.95 ml of triethylamine. 0.402 g of acetylsalicylic acid chloride was then added at a temperature of 0° C. The reaction mixture was heated to room temperature and then agitated for 12 hours. The mixture was then extracted by an aqueous solution saturated in NaCl. The organic phase treated in this manner was dried over MgSO₄. The solvent was evaporated and the residue was chromatographed on silica gel eluting with a 98:2 CH₂Cl₂/MeOH mixture. We obtained the stated compound (1.41 g) in the form of a white solid.

c) adenosine N-salicylyl

0.230 g of the compound obtained in b) was dissolved in 10 ml of a 7:3 methanol/water mixture, after which 0.232 g of K₂CO₃ was added. At the end of one hour, the mixture was washed several times with dichloromethane and the aqueous phase was concentrated. We obtained 0.156 g of a yellow solid.

The NMR spectrum was in agreement with the indicated structure.

Study of the Effects of NSAIDs Alone and Combined with Adenosine or Amp on Platelet Aggregation

Blood was collected on a citrate buffer from rats that had not received any treatment or from humans who had not received any treatment during the two weeks preceding collection of the blood sample. Platelet rich plasma was prepared by centrifugation using known methods.

Measurements were performed using an aggregometer marketed under the name “Chrono-log”. The measurements were processed and digitized using an “MP30 Biopac” data acquisition system.

a) Effects of AMP and Adenosine on Aggregation of Rat Platelets

We saw a rapid and noteworthy (80%) aggregation with rat platelets stimulated by ADP (20 μM).

When ADP was added in the same concentration in the presence of 2.5 mM or 5.0 mM AMP, the aggregation was only partial (40% and 20%, respectively), followed by a spontaneous disaggregation. Thus the action of AMP is dose dependent.

Comparable results were obtained when AMP (5 mM) was replaced by adenosine (4 mM).

We observed an immediate disaggregation when the rat platelets were stimulated by ADP (20 μM) followed by the addition 30 seconds later of AMP (10 mM).

In conclusion, adenosine and AMP can inhibit and reverse the aggregation of platelets induced by ADP.

b) Effects of NSAIDs Alone and Combined with AMP on the Aggregation of Rat Platelets

No significant inhibition of platelet aggregation was observed when we added sodium salicylate (7.5 mM) to the plasma followed by ADP (20 μM).

Practically no more aggregation was observed when we added ADP (20 μM) in the presence of sodium salicylate (7.5 mM) and AMP (2.5 mM).

Thus, the combination of AMP and sodium salicylate presents a potentiating synergic effect.

Similar effects were obtained when the sodium salicylate was replaced by aspirin (7.5 mM) or indomethacin (0.05 mM).

c) Effects of the Combination Aspirin+AMP on the Aggregation of Rat Platelets

The platelets were prepared from blood taken from rats treated 1 hour earlier by an intravenous injection of aspirin (2 mg/kg) and AMP (1 mg/kg).

The platelets were stimulated by ADP (10 and 20 μM).

With the dose of 10 and 20 μM ADP in control animals (not having received any prior treatment) we observed a platelet aggregation greater than 50% 3 minutes after the addition of ADP.

In the animals treated previously with aspirin and AMP, we observed a slight aggregation (circa 20%) which was reversed after approximately 3 minutes.

d) Effects of the Product of Example 1 on the Aggregation of Rat Platelets

Stimulation with ADP (20 μM) was performed in the presence of the product of example 1 (0.3 mM).

With the plasma that had not been previously treated, the maximum aggregation reached 55% with an initial slope of the curve of 0.99. In the presence of the product of Example 1 (added immediately after the addition of ADP), the maximum aggregation was 39% with an initial slope of 0.84. The result of the tests performed under these experimental conditions was that the reduction in aggregation was 30 to 50%.

When the product of Example I was added to the plasma 25 minutes prior to the stimulation by ADP, the maximum aggregation was less than 30%.

e) Effect of the Product of Example 1 on the Aggregation of Rat Platelets; Comparison with the Combination AMP+Aspirin

The platelets were stimulated by ADP (20 μM) either in the presence of the product of Example 1 (0.3 mM) or in the presence of aspirin (20 mM)+AMP (0.25 mM).

In the control animals, stimulation by ADP induced platelet aggregation to the level of 50%.

In the case of the plasma treated by the product of Example 1, the maximum aggregation reached 30% and then reversed itself.

In the case of the plasma having received aspirin and AMP, the aggregation reached 40% and then reversed itself.

The results show that the product of Example 1 is more active than the simple combination of aspirin and AMP.

f) Effects of the Product of Example 1 on Human Platelets

We studied the response of human platelets stimulated by ADP (20 μM) in the presence of the product of Example 1 (0.5 mM). In the controls, ADP induced a maximum platelet aggregation reaching 80%. In the case of the addition of ADP in the presence of the product of example 1, the maximum aggregation did not exceed 35% and began to reverse itself after 3 to 4 minutes.

In the case in which the platelets were stimulated by arachidonic acid (20 mM), we saw an aggregation of 70%. When the arachidonic acid was added in the presence of the product of Example 1 (0.005 mM), aggregation of the platelets was totally inhibited.

g) Response of Human Platelets Stimulated by Collagen

We studied the response of human platelets stimulated by collagen (0.19 mg/ml, Biodata). Two parameters were measured: the latency time (elapsed time between bringing the platelets into contact with collagen and the beginning of aggregation) and the maximum amplitude of aggregation.

The response to collagen in terms of maximum response was not modified by aspirin (0.6 mM), nor by sodium salicylate (2 mM), nor by AMP (0.6 mM), nor by the product of Example 1 (0.5 mM), nor by the combination of sodium salicylate (2 mM) and AMP (0.6 mM).

In contrast, at the dose indicated the product of Example 1 doubled the latency time that followed the bringing into contact of the platelets with collagen. The combination AMP (0.6 mM)+sodium salicylate (2 mM) was of an efficacy comparable to that of the product of Example 1 (0.5 mM). Sodium salicylate (2 mM) had no effect on the latency time. In the case of AMP (0.6 mM), the latency time was multiplied by circa 1.5.

Study of the Anti-Inflammatory Effect

The test was performed on male mice weighing 20 g. This protocol lasted 5 days. On day 1, we injected 3 cm³ of air in the dorsal part via the subcutaneous route. The resultant air pockets were reinflated on days 2, 3 and 4 with 1 cm³ of air. We administered via gastric gavage on day 3, day 4 and day 5, 1 hour prior to the induction of inflammation, either 1 ml of water (controls, n=14), or aspirin (100 mg/kg, n=5), or the product of example 1 (100 mg/kg, n=4), or the product of example 3 (100 mg/kg, n=5). We induced inflammation by injection of 1 ml of a 2% (weight/volume) suspension of carrageenan in PBS buffer into the air pocket. After 4 hours, the pockets were washed with 2 ml of PBS and the exudates were collected. Aliquot parts were diluted to 1:1 with a 0.01% solution of methylene blue in a PBS buffer. We then performed counting of the cells (principally neutrophils) whose accumulation was induced by the inflammation.

The results are summarized in Table 1. They show that the products of Examples 1 and 3 significantly decreased the number of leukocytes accumulated in the inflammatory exudate compared to the controls and compared to aspirin alone.

TABLE 1

| Product tested | Number of leukocytes | % |
|---|---|---|
| (doses in mg/kg/day) | (millions/ml) | inhibition |
| Controls | 9.57 ± 0.48 | |
| Aspirin (100) | 6.40 ± 0.77 | 33 |
| Product of example 1 (100) | 4.54 ± 0.55 | 53 |
| Product of example 3 (100) | 3.80 ± 0.80 | 60 |

Study of the Gastric Toxicity

This test was performed on Sprague Dawley rats (IFFA CREDO, l'Arbresle, France). The rats were in a fasting state the evening before the experiment (but drinking water was available ad libitum). The rats were treated via the subcutaneous route with indomethacin (20 mg/kg) or by gavage with 100 mg/kg of aspirin, or the product of Example 1, or the product of Example 3. Three hours later, the rats were sacrificed by a lethal dose of pentobarbital. The stomach was removed, opened along the greater curvature and rinsed with water. The gastric mucosa was photographed and digitized using a Macintosh G3 computer equipped with a program (NIH, USA) and a video card (Scion, USA). The stomach was fixed with buffered formalin and then processed with the conventional histological techniques for paraffin inclusion. The sections (5 μm) were then stained with hematoxylin-eosin-safranin.

Two observations were performed: macroscopic (after the stomach was collected, under the microscope) and histologic.

The stomachs of the rats treated with indomethacin presented pronounced macroscopic ulcerations confirmed by the histologic study. The stomachs of the rats treated by aspirin at this dose presented lesions in the form of petechiae or micro-ulcerations. This study showed that, compared to aspirin and indomethacin, the products of Examples 1 and 3 did not cause damage to the gastric wall in the rat in this in vivo model.

1. A method of treating inflammation or thrombosis comprising administering to a mammal a therapeutically effective amount of a composition comprising a purine and an NSAID as active ingredients.

2. The method according to claim 1, wherein the composition treats restenosis induced after angioplasty.

3. The method according to claim 1, wherein the purine is selected from the group consisting of adenosine, guanosine, inosine and corresponding nucleotides.

4. The method according to claim 1, wherein the purine is adenosine or AMP.

5. The method according to claim 1, wherein the NSAID is selected from the group consisting of derivatives of aminoarylcarboxylic acids, arylacetic acids, arylbutryic acids, arylcarboxylic acids, arylpropionic acids, salicylic acid, pyrazole derivatives, pyrazolone derivatives, thiazinecarboxamide derivatives, nitric esters, nitro derivatives and nitroso derivatives.

6. The method according to claim 1, wherein the NSAID is selected from the group consisiting of acetylsalicylic acid, salicylic acid, ibuprofen, naproxen, diclofenac, sulindac, celecoxib, rofecoxib, nabumetone and salts thereof.

7. The method according to claim 1, wherein the composition is administered in an amount of from about 10 to about 1000 mg of purine and from about 10 to about 3000 mg of NSAID.

8. The method according to claim 1, wherein the composition is in the form of a capsule, solution, emulsion, granule, gel, cream, powder, tablet, compressed tablet, unguent, injectable solution, injectable suspension, lyophilized powder, transdermal device or suppository.

9. The method according to claim 1, wherein one or more molecules of purine or a purine analogue are bound by covalence to one or more molecules of NSAID, optionally by a spacer arm intermediary.

10. The method according to claim 9, wherein the composition is formed by:
amidification of an amine function of the purine, or esterification of one or more alcohol functions of the purine by the NSAID with a carboxylic group, or
amidification of adenosine or AMP by the NSAID with a carboxylic group.

11. The method according to claim 1, wherein the NSAID is selected from the group consisting of acetylsalicylic acid, salicylic acid, diclofenac, naproxen, ibuprofen, sulindac and mefenamic acid.

12. The method according to claim 1, wherein the composition corresponds to formula I:
(A-)ₘ(X)ₚ(—B)ₙ (I)
wherein A is a residue of an NSAID molecule, B is a residue of a purine and X represents either a covalent bond between A and B, or a spacer arm linking at least one A residue with at least one B residue, the bonds between the spacer arm and the A and B residues being covalent bonds, m is a whole number ranging from 1 to 3, n is a whole number ranging from 1 to 3, and p represents zero or a whole number equaql at most to the larger of the numbers m and n, or the product is a salt of the corresponding to formula I.

13. The method according to claim 12, wherein the chemical groups creating the bond between A and B, or between A and X, or between X and B, are carboxylic ester, carboxylic amide, thiocarboxylic ester or thiocarboxylic amide groups.

14. The method according to claim 12, wherein the NSAID is selected from the group consisting of aspirin, salicylic acid, ibuprofen, naproxen, diclofenac and sulindac.

15. An anti-inflammatory or anti-thrombotic composition comprising one or more molecules of purine covalently linked to one or more molecules of NSAID, or optionally, by at least one intermediary spacer arm.

16. The composition according to claim 15, wherein the NSAID is an NSAID with a carboxylic group.

17. The composition according to claim 15, wherein the NSAID molecule is a nitric ester, a nitro derivation or nitroso derivative of an NSAID, or the purine is selected from the group consisting of adenosine and AMP.

18. The composition according to claim 15, wherein the covalent bond is provided by a carboxylic ester, carboxylic amide, thiocarboxylic ester or thiocarboxylic amide group.

19. The composition according to claim 15, which satisfies formula (I):
(A-)ₘ(X)ₚ(—B)ₙ (I)
wherein A is a residue of the NSAID molecule, B is a residue of the purine and X represents either a covalent bond between A and B, or a spacer arm linking at least one A residue with at least one B residue, the bonds between the spacer arm and the A and B residues being covalent bonds, m is a whole number ranging from 1 to 3, n is a whole number ranging from 1 to 3, and p represents zero or a whole number equal at most to the larger of the numbers m and n, or salts thereof.

20. The composition according to claim 19, wherein a bond between A and B, or between A and X, or between X and B is provided by a carboxylic ester, carboxylic amide, thiocarboxylic ester or thiocarboxylic amide group.

21. The composition according to claim 19, comprising products of amidification or esterification of adenosine or AMP by salicylic acid, acetylsalicylic acid, mefenamic acid, diclofenac, naproxen, ibuprofen or sulindac, or the nitric esters, nitro derivatives or nitroso derivatives.

22. A method of preventing inflammation or thrombosis comprising administering to a mammal a therapeutically effective amount of a composition comprising a purine and an NSAID as active ingredients.

23. The method according to claim 22, wherein the composition treats restenosis induced after angioplasty.

24. The method according to claim 22, wherein the purine is selected from the group consisting of adenosine, guanosine, inosine and corresponding nucleotides.

25. The method according to claim 22, wherein the purine is adenosine or AMP.

26. The method according to claim 22, wherein the NSAID is selected from the group consisting of derivatives of aminoarylcarboxylic acids, arylacetic acids, arylbutryic acids, arylcarboxylic acids, arylpropionic acids, salicylic acid, pyrazole derivatives, pyrazolone derivatives, thiazinecarboxamide derivatives, nitric esters, nitro derivatives and nitroso derivatives.

27. The method according to claim 22, wherein the NSAID is selected from the group consisiting of acetylsalicylic acid, salicylic acid, ibuprofen, naproxen, diclofenac, sulindac, celecoxib, rofecoxib, nabumetone and salts thereof.

28. The method according to claim 22, wherein the composition is administered in an amount of from about 10 to about 1000 mg of purine and from about 10 to about 3000 mg of NSAID.

29. The method according to claim 22 wherein the composition is in the form of a capsule, solution, emulsion, granule, gel, cream, powder, tablet, compressed tablet, unguent, injectable solution, injectable suspension, lyophilized powder, transdermal device or suppository.

30. The method according to claim 22, wherein one or more molecules of purine or a purine analogue are bound by covalence to one or more molecules of NSAID, optionally by a spacer arm intermediary.

31. The method according to claim 30, wherein the composition is formed by:
amidification of an amine function of the purine, or esterification of one or more alcohol functions of the purine by the NSAID with a carboxylic group, or
amidification of adenosine or AMP by the NSAID with a carboxylic group.

32. The method according to claim 22, wherein the NSAID is selected from the group consisting of acetylsalicylic acid, salicylic acid, diclofenac, naproxen, ibuprofen, sulindac and mefenamic acid.

33. The method according to claim 22, wherein the composition corresponds to formula I:
(A-)ₘ(X)ₚ(—B)ₙ (I)
wherein A is a residue of an NSAID molecule, B is a residue of a purine and X represents either a covalent bond between A and B, or a spacer arm linking at least one A residue with at least one B residue, the bonds between the spacer arm and the A and B residues being covalent bonds, m is a whole number ranging from 1 to 3, n is a whole number ranging from 1 to 3, and p represents zero or a whole number equaql at most to the larger of the numbers m and n, or the product is a salt of the corresponding to formula I.

34. The method according to claim 332, wherein the chemical groups creating the bond between A and B, or between A and X, or between X and B, are carboxylic ester, carboxylic amide, thiocarboxylic ester or thiocarboxylic amide groups.

35. The method according to claim 332, wherein the NSAID is selected from the group consisting of aspirin, salicylic acid, ibuprofen, naproxen, diclofenac and sulindac.
